(19) United States
(12) Patent Application Publication
(10) Pub. No.: US 2008/0019957 A1
(43) Pub. Date: Jan. 24, 2008

(54) TOPICALLY APPLIED PROBUCOL-CONTAINING AGENT WITH PROTECTIVE AND REGENERATIVE EFFECT

(76) Inventors: Birgit Neudecker, Munchen (DE); Falko Diedrich, Munchen (DE); Eberhard Wieland, Stuttgart (DE)

Correspondence Address: Joseph W. Holland;HOLLAND LAW OFFICE PLLC, P.O. Box 1840, Boise, ID, 83701-1840, US

(21) Appl. No.: 11/780,662

(22) Filed: Jul. 20, 2007

Related U.S. Application Data
(60) Provisional application No. 60/807,899, filed on Jul. 20, 2006.

(51) Int. Cl. A61K 31/10; A61K 31/122; A61K 31/192; A61K 31/194; A61K 31/34; A61K 31/35; A61K 31/352; A61K 31/355; A61K 31/385; A61K 31/52; A61K 31/6615; A61K 31/726; A61K 31/728; A61K 36/484; A61K 36/605; A61K 36/82; A61K 36/87; A61K 38/02; A61K 38/44; A61Q 17/04; A61Q 19/08

(52) U.S. Cl. 424/94.4; 424/729; 424/757; 424/766; 424/769; 514/143; 514/18; 514/263.23; 514/440; 514/453; 514/458; 514/460; 514/474; 514/54; 514/558; 514/568; 514/689; 514/712

(57) ABSTRACT

A method of treating skin changes and conditions in human skin including applying a topical preparation that includes an active agent including probucol, a derivative of probucol or a combination of probucol and a derivative of probucol.

### RELATED APPLICATIONS

This application claims the priority of Provisional Application Ser. No. 60/807,899 entitled Topically Applied Probucol-Containing Agent with Protective and Regenerative Effect, filed Jul. 20, 2006, the content of said application being incorporated herein by reference.

The present invention in general relates to human skin, and to cosmetic and topical dermatological preparations containing an effective amount of probucol and/or its derivatives. The present invention relates in particular to cosmetic preparations having effective protection from damaging oxidation processes in the skin, but also for the protection of cosmetic preparations themselves, or for the protection of the constituents of cosmetic preparations from damaging oxidation processes. Furthermore, the present invention relates to cosmetic and dermatological preparations containing an effective amount of probucol and/or its derivatives, which beyond supporting vesicular breathing/cellular respiration or stabilization of the mitochondrial membranes, promote the regeneration and vitality of skin cells.

The present invention also relates to anti-oxidants, and here preferably those which are used in skin-care cosmetic or dermatological preparations. The invention also relates particularly to cosmetic and dermatological preparations containing such anti-oxidants in combination with probucol and/or its derivatives. Furthermore, the present invention relates to cosmetic and dermatological preparations for the prophylaxis and treatment of cosmetic and dermatological skin changes, such as for example skin aging, and here in particular skin aging produced by oxidative or degenerative processes.

### BACKGROUND

Human skin is susceptible to oxidative stress due to various causes, such as external environmental stressors including exposure to ultraviolet radiation, air pollution, cigarette smoke, chemicals, cosmetics, drugs, ozone and even oxygen itself, as well as to internal stress due to natural biological processes.

For example, the damaging effect of the ultraviolet (UV) part of solar radiation on the skin is well recognized. UV radiation is known to be the predominant cause of premature aging of the skin. UV radiation may lead to photochemical reactions, wherein then the photochemical reaction products intervene in the skin mechanism. Predominantly such photochemical reaction products are free-radical compounds, for example hydroxy radicals. Also, undefined free-radial photoproducts, which are produced in the skin itself, may trigger uncontrolled side reactions due to their high reactivity. Furthermore, lipid peroxidation products, such as for example hydroperoxides and aldehydes, are produced. Free-radical chain reactions may be triggered, resulting in skin damage.

Moreover, UV radiation is ionizing radiation. Hence, there is the risk that ionic species are produced on UV exposure, which species in turn are able to intervene oxidatively in the biochemical processes.

In additional to externally generated oxidative stress, internally generated oxidative stress may occur as a natural by-product of cellular energy production. Both internal and external oxidative stress leads to inflammatory pathways mediated by the formation of free radicals (molecules with unpaired electrons that are highly reactive) that, left unchecked, can cause severe cellular damage to skin cell membranes, lipids, proteins and DNA. The superoxide radical, a natural by-product of metabolic energy production, causes serious deleterious effects to living cells if not quenched, neutralized or reduced almost immediately after production. It is known that lipid peroxidation is a major problem in biological systems. Protecting against cell membrane oxidation is of paramount importance in living biological systems since the cell membrane is the cell's first line of defense against oxidation

The lifetime summation of damage caused by run-away free radicals is one of the main theories of aging, “the damage accumulation theory of aging”. There is therefore a high interest in modern medicine regarding the use of antioxidants, substances that scavenge and eliminate free radicals, to counter the deleterious effects (i.e., aging) of the free radical mediated inflammatory pathways caused by oxidative stress.

Predominantly such photochemical reaction products are free-radical compounds, for example hydroxy radicals. Also, undefined free-radial photoproducts, which are produced in the skin itself, may trigger uncontrolled side reactions due to their high reactivity. However, singlet oxygen, a non-free-radical excited state of the oxygen molecule, may occur on UV irradiation, likewise short-lived epoxides and many others. Singlet oxygen, for example is characterized with respect to the normally existing triplet oxygen (free-radical base state) by increased reactivity. Nevertheless, excited, reactive (free-radical) triplet states of the oxygen molecule also exist. Furthermore, there is the occurrence of lipid peroxidation products, such as for example hydroperoxides and aldehydes, wherein first in turn free-radical chain reactions may be triggered and to which overall cytotoxic properties have to be ascribed.

Oxidative damage to the skin and its more detailed causes are discussed in J. Fuchs et al., “Skin Diseases Associated with Oxidative Injury” in “Oxidative Stress in Dermatology”, 1983, pp. 323 et seq., which is hereby incorporated by reference herein.

Antioxidants are substances, to include free radical absorbers or scavengers, that prevent oxidation processes, including oxidation of a molecule such as a lipid, lipoprotein, protein or DNA, or autooxidation of fats containing unsaturated compounds. Various antioxidants are used in the field of cosmetics and pharmacy. These include, for example tocopherol, idebenone, kinetin, ubiquinone, ascorbic acid, lipoic acid sesamol, colic acid derivatives, butylhydroxy anisole and butylhydroxy toluene. Antioxidants can thus help protect human cells, such as skin cells, from both externally and internally generated oxidative stress.

U.S. Pat. No. 4,144,325 describes using vitamin E in sunscreen formulas. U.S. Pat. No. 6,756,045 describes treating skin changes, for example involving oxidative processes, using idebenone.

Topical application of antioxidants is used to optimize the cutaneous antioxidative capacity and to limit skin damage. In vitro and in vivo studies have demonstrated antioxidative and photoprotective properties of antioxidants. See K. Scharffetter-Kochanek et al., “Photoaging of the skin from phenotype to mechanisms”, Exp Gerontol 2000, 35:307-316; and J. Wenk et al., “UV-induced oxidative stress and photoaging”, Curr Probl Dermatol 2001, 29:83-94, both of which are hereby incorporated by reference herein.

### SUMMARY OF THE INVENTION

One object of the present invention is to provide active ingredients or preparations containing such active ingredients which when used may reduce the damage to the skin by oxidative influence, if not completely prevent it and which additionally show a regenerating and vitalizing effect on aging, stressed or damaged skin by supporting vesicular breathing, stabilization of mitochondrial membranes and anti-apoptotic properties.

The present invention provides a method for treating, preventing and/or reducing oxidation processes in human skin, comprising applying a topical preparation comprising an amount of an agent effective to prevent and/or reduce an oxidation process in human skin, the agent comprising probucol or a derivative of probucol.

The present invention also provides a method for preventing and/or reducing skin changes and/or damage due to oxidation processes in human skin, comprising ingesting an oral preparation comprising an amount of an agent effective to prevent and/or reduce skin changes and/or damage due to oxidation processes in human skin, the agent comprising probucol or a derivative of probucol.

The present invention also provides a method for preventing and/or reducing skin changes and/or damage due to oxidation processes in human skin, comprising applying a topical preparation comprising an amount of an agent effective to treat, prevent and/or reduce skin changes and/or damage due to oxidation processes in human skin, the agent comprising probucol or a derivative of probucol.

The present invention also provides a method for preventing and/or reducing oxidation of a component of a skin preparation, comprising providing an amount of an agent effective to prevent and/or reduce the oxidation of the component, the agent comprising probucol or a derivative of probucol.

The use of probucol and/or its derivatives as anti-oxidant is thus of the invention. The use of probucol and/or its derivatives as anti-oxidants for the stabilization of cosmetic or dermatological preparations, which contain as additive either vitamin A and/or its derivatives (for example all-E-retinoic acid, 9-Z-retinoic acid, 13-Z-retinoic acid, retinal, retinyl ester), vitamin B and/or its derivatives, vitamin C and/or its derivatives and vitamin E and/or its derivatives (for example alpha-tocopherol acetate) individually or in combination, is thus likewise of the invention. The stabilizing effect thus relates to both smell and color and in particular to the active ingredient content of the preparation.

The derivative of probucol may be, for example, [mono[4-[[1-[[3,5-bis(1,1-dimethylethyl)-4-hydroxyphenyl]thio]-11-methylethyl]thio]-2,6-bis(1,1-dimethylethyl)phenyl]ester] (butanedioc acid) also named AGI-1067, [2-[4-[[1-[[3,5-bis(1,1-dimethylethyl)-4-hydroxyphenyl]thio]-1-methylethyl]thio]-2,6-bis(1,1-dimethylethyl)phenoxy]acetic acid] also named AGIX-4207, 4,4′-[methylenebis(thio)]bis](1,1-dimethylethyl)]-phenol also named MDL 29311, or 8-(3-(3,5-di-tert-butyl-4-hydroxyphenylthio)propyl)-1-oxa-2-oxo-3,8-diazaspiro(4.5)decane also named S 12340.

### DETAILED DESCRIPTION

Probucol, 2,2-Propdiyl-bis(4-thio-2,6-di-tert-butylphenole), has the following structure:

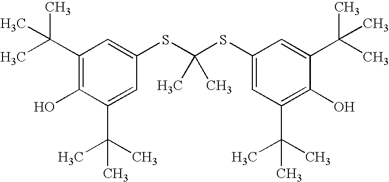

Laboratory results have demonstrated probucol and/or its derivatives to be a potential antioxidant.

Probucol used according to the invention and its derivatives or cosmetic or dermatological preparations containing them

act better as anti-oxidant

act better as free-radical absorbers/scavengers

better prevent damage of lipids, DNA and proteins

act better against skin aging and wrinkle formation

better protect the skin against photo-reactions

better prevent inflammatory skin reactions

would better start regeneration processes in the skin than active ingredients and preparations of the state of the art.

The use of probucol and/or its derivatives is therefore of the invention. The use of other derivatives of probucol is of course also of the invention.

The use of probucol and/or its derivatives as anti-oxidant and its use for combating and/or prophylaxis of skin aging caused by oxidative stress and inflammatory reactions are likewise of the invention. The use of probucol and/or its derivatives as anti-oxidants for the stabilization of cosmetic or dermatological preparations, which contain as additive either vitamin A and/or its derivatives (for example all-E-retinoic acid, 9-Z-retinoic acid, 13-Z-retinoic acid, retinal, retinyl ester), vitamin B and/or its derivatives, vitamin C and/or its derivatives and vitamin E and/or its derivatives (for example alpha-tocopherol acetate) individually or in combination, is thus likewise of the invention. The stabilising effect thus relates to both smell and color and in particular to the active ingredient content of the preparation.

Furthermore, the use of probucol and its derivatives as an agent for stabilization of mitochondrial membranes with additional anti-apoptotic effect in skin cells and its use for the regeneration and revitalization of aging, stressed or damaged skin, is therefore also of the invention.

The cosmetic or dermatological formulations of the invention may be composed as is conventional and serve for the treatment, care and cleansing of the skin and as a make-up product in decorative cosmetics. They preferably contain 0.0001 wt. % to 30 wt. %, preferably 0.05 wt. % to 5 wt. %, in particular 0.1-2.0 wt. %, based on the total weight of the agent, of probucol and/or its derivatives.

For administration, the cosmetic and dermatological preparations of the invention are applied to the skin in adequate quantity in the manner conventional for cosmetics.

Cosmetic and dermatological preparations of the invention may exist in various forms. Hence, they may be, for example a solution, an anhydrous preparation, an emulsion or microemulsion of the type water-in-oil (W/O) or of the type oil-in-water (O/W), a multiple emulsion, for example of the type water-in-oil-in-water (W/O/W), a gel, a solid stick, an ointment or even an aerosol. It is also advantageous to administer probucol and/or its derivatives in encapsulated form, for example in collagen matrices and other conventional encapsulation materials, for example as cellulose encapsulations, in gelatine, wax matrices or liposomally encapsulated.

It is also possible and advantageous within the scope of the present invention to add probucol and/or its derivatives to aqueous systems or surfactant preparations for cleansing the skin.

The use of probucol for the protection of the skin from oxidative stress is therefore also regarded as an advantageous embodiment of the present invention, in particular this use of probucol in washing formulations.

The cosmetic and dermatological preparations of the invention may contain cosmetic auxiliaries, as are used conventionally in such preparations, for example preservatives, bactericides, perfumes, substances for preventing foaming, dyestuffs, pigments which have a coloring effect, thickening agents, surfactant substances, emulsifiers, softening, moisturizing and/or moisture-retaining substances, fats, oils, waxes or other conventional constituents of a cosmetic or dermatological formulation, such as alcohols, polyols, polymers, foam stabilizers, electrolytes, organic solvents or silicone derivatives.

In particular, probucol and its derivatives may also be combined according to the invention with other anti-oxidants and/or free-radical absorbers.

All anti-oxidants which are suitable or conventional for cosmetic and/or dermatological applications may be used according to the invention as favorable anti-oxidants.

The anti-oxidants are advantageously selected from the group consisting of amino acids (for example glycine, histidine, tyrosine, tryptophan) and their derivatives, imidazoles (for example urocanic acid) and their derivatives, peptides, such as D,L-carnosine, D-carnosine, L-carnosine and their derivatives (for example anserine), carotinoids, carotenes (for example alpha-carotene, beta-carotene, lycopene) and their derivatives, chlorogenic acid and its derivatives, lipoic acid and its derivatives (for example dihydrolipoic acid), aurothioglucose, propylthiouracil and other thiols (for example thioredoxin, glutathione, cysteine, cystine, cystamine and their glycosyl, N-acetyl, methyl, ethyl, propyl, amyl, butyl and lauryl, palmitoyl, oleyl, gamma-linoleyl, cholesteryl and glyceryl esters) and their salts, dilauryl thiodipropionate, distearyl thiodipropionate, thiodipropionic acid and their derivatives (esters, ethers, peptides, lipids, nucleotides, nucleosides and salts) and sulphoximine compounds (for example buthionine sulphoximines, homocysteine sulphoximine, buthionine sulphones, pentathionine sulphoximine, hexathionine sulphoximine, heptathionine sulphoximine) in very low, acceptable doses (for example pmole to μmoles/kg), also (metal) chelating agents (for example alpha-hydroxy fatty acids, palmitic acid, phytic acid, lactoferrin), alpha-hydroxy acids (for example citric acid, lactic acid, malic acid, mandelic acid), humic acid, colic acid, colic extracts, bilirubin, biliverdin, EDTA, EGTA and their derivatives, unsaturated fatty acids and their derivatives (for example gamma-linolenic acid, linolic acid, oleic acid), folic acid and their derivatives, ubiquinone and ubiquinol and their derivatives, vitamin C and derivatives (for example ascorbyl palmitate, Mg-ascorbyl phosphate, ascorbyl acetate), tocopherols and derivatives (for example vitamin E acetate), vitamin B and derivatives, idebonone and derivatives, vitamin A and derivatives (for example vitamin A palmitate) and coniferyl benzoate of benzoin resin, rutinic acid and their derivatives, butylhydroxy toluene, butylhydroxy anisole, nordihydroguaiacic acid, nordihydroguaiaretic acid, trihydroxybutyrophenone, uric acid and its derivatives, mannose and its derivatives, sesamol, sesamolin, zinc and its derivatives (for example ZnO, ZnSO₄), selenium and its derivatives (for example selenium methionine), stilbenes and their derivatives (for example stilbene oxide, trans-stilbene oxide) and the suitable derivatives of the invention (salts, esters, ethers, sugars, nucleotides, nucleosides, peptides and lipids) of these said active ingredients.

The quantity of the aforementioned anti-oxidants (one or more compounds) in the preparations is preferably 0.0001 wt. % to 30 wt. %, particularly preferably 0.05 wt. % to 20 wt. %, in particular 1 B 10 wt. %, based on the total weight of the preparation.

Provided vitamin E and/or its derivatives represent the additional anti-oxidant(s), it is advantageous to select their particular concentration from the range from 0.0001 B 20 wt. %, based on the total weight of the formulation.

Provided vitamin A or vitamin A derivatives or carotenes or their derivatives represent the additional anti-oxidant(s), it is advantageous to select their particular concentrations from the range from 0.0001 B 10 wt. %, based on the total weight of the formulation.

Emulsions of the invention are advantageous and contain, for example the said fats, oils, waxes and other adipoids, and water and an emulsifier, as is used conventionally for such a type of formulation.

The lipid phase may advantageously be selected from the following substance group:

mineral oils, mineral waxes;
oils, such as triglycerides of capric or caprylic acid, also natural oils, such as for example castor oil;
fats, waxes and other natural and synthetic adipoids, preferably esters of fatty acids with alcohols of low C number, for example with isopropanol, propylene glycol or glycerine, or esters of fatty alcohols with alkane acids of low C number or with fatty acids;
alkyl benzoates;
silicone oils, such as dimethylpolysiloxanes, diethylpolysiloxanes, diphenylpolysiloxanes and mixtures thereof.

The oil phase of the emulsions, oleogels or hydrodispersions or lipodispersions within the scope of the present invention is advantageously selected from the group of esters of saturated and/or unsaturated, branched and/or unbranched alkane carboxylic acids of chain length from 3 to 30 C atoms and saturated and/or unsaturated, branched and/or unbranched alcohols of chain length from 3 to 30 C atoms, from the group of esters from aromatic carboxylic acids and saturated and/or unsaturated, branched and/or unbranched alcohols of chain length from 3 to 30 C atoms. Such ester oils may then advantageously be selected from the group isopropyl myristate, isopropyl palmitate, isopropyl stearate, isopropyl oleate, n-butyl stearate, n-hexyl laurate, n-decyloleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate and synthetic, semi-synthetic, and natural mixtures of such esters, for example jojoba oil.

Furthermore, the oil phase may advantageously be selected from the group of branched and unbranched hydrocarbons and waxes, silicone oils, dialkyl ethers, the group of saturated or unsaturated, branched or unbranched alcohols, and fatty acid triglycerides, namely the triglycerine esters of saturated and/or unsaturated, branched and/or unbranched alkane carboxylic acids of chain length from 8 to 24, in particular 12-18, C atoms. The fatty acid triglycerides may advantageously be selected, for example from the group of synthetic, semi-synthetic and natural oils, for example olive oil, sunflower oil, soybean oil, peanut oil, rape-seed oil, almond oil, palm oil, coconut oil, palm kernel oil and the like.

Also any mixtures of such oil and wax components can be used advantageously within the scope of the present invention. It may also optionally be advantageous to use waxes, for example cetyl palmitate, as the single lipid component of the oil phase.

The oil phase is advantageously selected from the group 2-ethylhexyl isostearate, octyl dodecanol, isotridecyl isononanoate, isoeicosane, 2-ethylhexyl cocoate, C₁₂₋₁₅ alkyl benzoate, capryl-capric acid triglyceride, dicaprylyl ether. Mixtures of C₁₂₋₁₅ alkyl benzoate and 2-ethylhexyl isostearate, mixtures of C₁₂₋₁₅ alkyl benzoate and isotridecyl isononanoate and mixtures of C₁₂₋₁₅ alkyl benzoate, 2-ethylhexyl isostearate and isotridecyl isononanoate are particularly advantageous.

Of the hydrocarbons, paraffin oil, squalane and squalene can be used advantageously within the scope of the present invention.

The oil phase may advantageously also contain cyclic or linear silicone oils or may consist completely of such oils, but wherein it is preferable, apart from the silicone oil or the silicone oils, to use an additional amount of other oil phase components.

Cyclomethicone (octamethylcyclotetrasiloxane) is advantageously employed as silicone oil to be used according to the invention. However, other silicone oils should also advantageously be used within the scope of the present invention, for example hexamethylcyclotrisiloxane, polydimethylsiloxane, poly(methylphenylsiloxane).

Mixtures of cyclomethicone and isotridecyl isononanoate, of cyclomethicone and 2-ethylhexyl isostearate, are also particularly advantageous.

The aqueous phase of the preparations of the invention contains optionally advantageously alcohols, diols or polyols of low C number, and their ethers, preferably ethanol, isopropanol, propylene glycol, glycerine, ethylene glycol, ethylene glycol monoethyl or monobutyl ether, propylene glycol monomethyl, monoethyl or monobutyl ether, diethylene glycol monomethyl or monoethyl ether and analogous products, also alcohols of low C number, for example ethanol, isopropanol, 1,2-propane diol, glycerineand in particular one or more thickening agents, which may advantageously be selected from the group silicon dioxide, aluminium silicates, polysaccharides or their derivatives, for example hyaluronic acid, xanthan gum, hydroxypropylmethylcellulose, particularly advantageously from the group of polyacrylates, in each case individually or in combination.

Mixtures of the above-mentioned solvents are used in particular. For alcoholic solvents, water may be a further constituent.

Emulsions of the invention are advantageous and contain, for example the said fats, oils, waxes and other adipoids, and water and an emulsifier, as is used conventionally for such a type of formulation.

Gels according to the invention conventionally contain alcohols of low C number, for example ethanol, isopropanol, 1,2-propane diol, glycerine and water or an above-mentioned oil in the presence of a thickening agent, which for oily-alcoholic gels is preferably silicon dioxide or an aluminium silicate, for aqueous-alcoholic or alcoholic gels is preferably a polyacrylate.

The conventional known highly volatile, liquefied propellants, for example hydrocarbons (propane, butane, isobutane), which may be used alone or mixed with one another, are suitable as propellants for preparations which can be sprayed from aerosol containers according to the invention. Compressed air can also advantageously be used.

Preparations of the invention may also advantageously contain substances which absorb UV radiation in the UVB range, wherein the total quantity of filter substances is, for example 0.1 wt. % to 30 wt. %, preferably 0.5 to 10 wt. %, in particular 1.0 to 6.0 wt. %, based on the total weight of the preparations, in order to provide cosmetic preparations which protect the skin from the entire range of ultraviolet radiation. They may also serve as sunscreen agents for the skin.

If the preparations of the invention contain UVB filter substances, they may be oil-soluble or water-soluble. According to the invention, advantageous oil-soluble UVB filters are, for example:

mineral oils, mineral waxes;
oils, such as triglycerides of capric or caprylic acid, also natural oils, such as for example castor oil;
fats, waxes and other natural and synthetic adipoids, preferably esters of fatty acids with alcohols of low C number, for example with isopropanol, propylene glycol or glycerine, or esters of fatty alcohols with alkane acids of low C number or with fatty acids;
alkyl benzoates;
silicone oils, such as dimethylpolysiloxanes, diethylpolysiloxanes, diphenylpolysiloxanes and mixtures thereof.
3-benzylidene camphor derivatives, preferably 3-(4-methylbenzylidene) camphor, 3-benzylidene camphor;
4-aminobenzoic acid derivatives, preferably (2-ethylhexyl) 4-(dimethylamino)benzoate, amyl 4-(dimethylamino)benzoate;
esters of cinnamic acid, preferably (2-ethylhexyl) 4-methoxycinnamate, isopentyl 4-methoxycinnamate;
esters of salicylic acid, preferably (2-ethylhexyl)salicylate, (4-isopropyl-benzyl)salicylate, homomentyl salicylate,
derivatives of benzophenone, preferably 2-hydroxy-4-methoxybenzophenone, 2-hy-droxy-4-methoxy-4=-methylbenzophenone, 2,2=-dihydroxy-4-methoxybenzophenone;
esters of benzylidenemalonic acid, preferably di(2-ethylhexyl) 4-methoxybenzylidenemalonate, -2,4,6-trianilino(p-carbo-2=-ethyl-=hexyloxy)-1,3,5-triazine.

Advantageous water-soluble UVB filters are, for example:

salts of 2-phenylbenzimidazole-5-sulphonic acid, such as its sodium, potassium or its triethanol-ammonium salt, and the sulphonic acid itself;
sulphonic acid derivatives of benzophenones, preferably 2-hydroxy-4-methoxybenzophenone-5-sulphonic acid and their salts;
sulphonic acid derivatives of 3-benzylidene camphor, such as for example 4-(2-oxo-3-bornylidene-methyl)benzene sulphonic acid, 2-methyl-5-(2-oxo-3-bornylidene-methyl)sulphonic acid and their salts as well as 1,4-di(2-oxo-10-sulpho-3-bornylidene-methyl)benzene and its salts (the corresponding 10-sulphato compounds, for example the corresponding sodium, potassium or triethanol; -ammonium salt), also designated as benzene-1,4-di(2-oxo-1-bornylidene-methyl)-10-sulphonic acid.

The list of the said UVB filters, which may be used in combination with the active ingredient combinations of the invention, should of course not be limiting.

The object of the invention is also the use of a combination of probucol and/or its derivatives with at least one UVB filter as anti-oxidant or the use of a combination of probucol and/or its derivatives with at least one UVB filter as anti-oxidant in a cosmetic or dermatological preparation.

It may also be advantageous to combine probucol and/or its derivatives with UVA filters, which hitherto are conventionally present in cosmetic preparations. These substances are preferably derivatives of dibenzoylmethane, in particular 1-(4=-tert.butylphenyl)-3-(4=-methoxyphenyl)propane-1,3-dione and 1-phenyl-3-(4=-isopropyl-phenyl)propane-1,3-dione. These combinations or preparations which contain these combinations are also an object of the invention. The quantities used for the UVB combination may be used.

The object of the invention is also the use of a combination of probucol and/or its derivatives with at least one UVA filter as anti-oxidant or the use of a combination of the active ingredient combinations of the invention with at least one UVA filter as anti-oxidant in a cosmetic or dermatological preparation.

The object of the invention is also the use of a combination of probucol and/or its derivatives with at least one UVA filter and at least one UVB filter as anti-oxidant or the use of a combination of probucol and/or its derivatives with at least one UVA filter and at least one UVB filter as anti-oxidant in a cosmetic or dermatological preparation.

Cosmetic and dermatological preparations having an effective amount of probucol and/or its derivatives may also contain inorganic pigments, which are used conventionally in cosmetics to protect the skin from UV rays. They are oxides of titanium, zinc, zirconium, silicon, manganese, cerium and mixtures thereof, and modifications in which the oxides are the active agents. They are particularly preferably pigments based on titanium dioxide.

These combinations of UVA filters and pigment or preparations containing this combination are also an object of the invention. The quantities mentioned for the above combinations may be used.

Cosmetic preparations which are a skin-cleansing agent or shampooing agent preferably contain at least one anionic, non-ionic or amphoteric surfactant substance, or also mixtures of such substances, probucol and/or its derivatives in aqueous medium and auxiliaries, as are used conventionally therefor. The surfactant substance or the mixtures of these substances may be present in the shampooing agent in a concentration between 1 wt. % and 50 wt. %.

These cosmetic or dermatological preparations may also be aerosols having the auxiliaries conventionally used therefor.

Aqueous cosmetic cleansing agents of the invention or low-water or anhydrous cleansing agent concentrates intended for aqueous cleansing may contain anionic, nonionic and/or amphoteric surfactants, for example traditional soaps, for example fatty acid salts of sodium alkyl sulphates, alkyl ether sulphates, alkane and alkyl benzene sulphonates

sulphoacetates
sulphobetaines
sarcosinates
amidosulphobetaines
sulphosuccinates
sulphosuccinic acid semi-esters
alkyl ether carboxylates
protein-fatty acid condensates
alkylbetaines and amidobetaines
fatty acid alkanol amides
polyglycol ether derivatives.

Cosmetic preparations which are cosmetic cleansing preparations for the skin, may be present in liquid or solid form. In addition to probucol and/or its derivatives, they preferably contain at least one anionic, non-ionic or amphoteric surfactant substance or mixtures thereof, if required one or more electrolytes and auxiliaries, as are used conventionally therefor. The surfactant substance may be present in the cleansing preparations in a concentration between 0.001 and 99.999 wt. %, based on the total weight of the preparations.

Cosmetic preparations which are a shampooing agent, in addition to a effective amount of probucol and/or its derivatives, preferably contain an anionic, non-anionic or amphoteric surfactant substance or mixture thereof, optionally an electrolyte of the invention and auxiliaries, as are used conventionally therefor. The surfactant substance may be present in the shampooing agent in a concentration between 0.001 wt. % and 99.999 wt. %.

The compositions of the invention contain, apart from the afore-mentioned surfactants, water and optionally the additives which are conventional in cosmetics, for example perfume, thickener, dyestuffs, deodorants, antimicrobial materials, back-fatting agents, complexing and sequestering agents, pearlescent agents, plant extracts, vitamins and/or their derivatives, active ingredients and the like.

The present invention also includes a cosmetic process for protecting the skin and the hair from oxidative or photooxidative processes, which is characterized in that a cosmetic agent, which contains an effective concentration of probucol and/or its derivatives, is applied to the skin or hair in adequate quantity.

Likewise, the present invention also includes a process for protecting cosmetic or dermatological preparations from oxidation or photooxidation, wherein these preparations, for example preparations for treating and caring for the hair are, in particular hair lacquers, shampooing agents, also make-up products, such as for example nail varnishes, lipsticks, foundations, washing and showering preparations, creams for treating or caring for skin or are all other cosmetic preparations, the constituents of which may bring with them stability problems due to oxidation or photooxidation on storage, characterized in that the cosmetic preparations have an effective amount of probucol and/or its derivatives.

The quantity of probucol and/or its derivatives in these preparations is preferably 0.0001 B 30 wt. %, preferably 0.05 B 5 wt. %, in particular 0.1 B 2.0 wt. %, based on the total weight of the preparations.

The object of the invention is also the process for producing the cosmetic agents of the invention, which is characterized in that active ingredient combinations of the invention are incorporated into cosmetic and dermatological formulations in a manner known per se.

The examples below are to illustrate the present invention without restricting it. All quantity details, proportions and percentage details are, unless otherwise stated, based on the weight and the total quantity or on the total weight of the preparations.

### EXAMPLE 1

O/W lotion

| | Wt. % |
|---|---|
| Paraffin oil (DAB 9) | 8.00 |
| Petrolatum | 4.00 |
| Octylmethoxy cinnamate | 5.00 |
| Isopropyl palmitate | 3.00 |
| Glycerine | 3.00 |
| Cetylstearyl alcohol | 2.00 |
| Butylmethoxy dibenzoylmethane | 1.00 |
| PEG-40 castor oil | 0.50 |
| Sodium cetylstearyl sulphate | 0.50 |
| Probucol | 0.50 |
| Hyaluronate | 0.50 |
| Sodium carbomer | 0.40 |
| Alpha-tocopherol | 0.20 |
| Preservatives, dyestuffs, perfume | q.s. |
| Water | ad 100.00 |

### EXAMPLE 2

O/W cream

| | Wt. % |
|---|---|
| Paraffin oil (DAB 9) | 7.00 |
| Avocado oil | 4.00 |
| Sodium lactate | 3.00 |
| Glycerine | 3.00 |
| Glyceryl monostearate | 2.00 |
| Titanium dioxide | 1.00 |
| Probucol | 0.50 |
| Hyaluronate | 0.50 |
| Preservatives, dyestuffs, perfume | q.s. |
| Water | ad 100.00 |

### EXAMPLE 3

W/O cream

| | Wt. % |
|---|---|
| Paraffin oil (DAB 9) | 10.00 |
| Caprylic acid/capric acid triglyceride | 5.00 |
| Buxus Chinensis | 5.00 |
| Avocado oil | 4.00 |
| PEG-40 hydrogenated castor oil | 4.00 |
| 1,2-Propylene glycol | 3.00 |
| Vaseline | 3.00 |
| Probucol | 1.00 |
| Alpha-tocopherol acetate | 1.00 |
| Retinol 10 CM | 1.00 |
| Paraben | 0.50 |
| Probucol | 0.50 |
| Idebenone | 0.50 |
| Hyaluronate | 0.50 |
| Alpha-tocopherol | 0.50 |
| Preservatives, dyestuffs, perfume | q.s. |
| Water | ad 100.00 |

### EXAMPLE 4

Lip care stick

| | Wt. % |
|---|---|
| Petrolatum | 40.00 |
| Ceresin | 8.00 |
| Hydrogenated castor oil | 4.00 |
| Beeswax | 4.00 |
| Carnauba wax | 2.00 |
| Probucol | 0.50 |
| β-Carotene | 0.10 |
| Preservatives, dyestuffs, perfume | q.s. |
| Paraffin oil | ad 100.00 |

### EXAMPLE 5

Lip care stick

| | Wt. % |
|---|---|
| Petrolatum | 40.00 |
| Isopropyl lanolate | 10.00 |
| Beeswax, bleached | 9.00 |
| Acetylated lanolin | 4.00 |
| Carnauba wax | 4.00 |
| Glycerine | 3.00 |
| Probucol | 0.50 |
| Alpha-tocopherol acetate | 0.10 |
| Preservatives, dyestuffs, perfume | q.s. |
| Paraffin oil | ad 100.00 |

### EXAMPLE 6

Hair tonic

| | Wt. % |
|---|---|
| Ethanol | 40.00 |
| Probucol | 0.50 |
| Alpha-tocopherol acetate | 0.50 |
| PEG-40 hydrogenated castor oil | 0.20 |
| Diisopropyl adipate | 0.10 |
| Preservatives, dyestuffs, perfume | q.s. |
| Water | ad 100.00 |

### EXAMPLE 7

Liposome-containing gel

| | Wt. % |
|---|---|
| Lecithin | 6.00 |
| Sorbitol | 3.00 |
| Hydrolysed collagen | 2.00 |
| Xanthan gum | 1.40 |
| Sodium citrate | 0.50 |
| Sodium PCA | 0.50 |
| Probucol | 0.50 |
| Glycine | 0.20 |
| Urea | 0.20 |
| Alpha-tocopherol | 0.20 |
| Biotin | 0.08 |
| Preservatives, dyestuffs, perfume | q.s. |
| Water | ad 100.00 |

### EXAMPLE 8

Sunscreen emulsion

| | Wt. % |
|---|---|
| Octylmethoxy cinnamate | 5.00 |
| Castor oil | 4.00 |
| Glycerine | 3.00 |
| Octyl stearate | 3.00 |
| Laurylmethicone copolyol | 2.00 |
| Cyclomethicone | 2.00 |
| Cetylstearyl alcohol | 1.80 |
| Na₃HEDTA | 1.50 |
| Glycerol lanolate | 1.00 |
| Butylmethoxy dibenzoylmethane | 1.00 |
| Probucol | 0.50 |
| PEG-40 hydrogenated castor oil | 0.30 |
| Sodium cetylstearyl sulphate | 0.30 |
| Acrylamide/sodium acrylate copolymer | 0.30 |
| Alpha-tocopherol | 0.20 |
| Caprylic acid/capric acid triglyceride | 0.10 |
| Preservatives, dyestuffs, perfume | q.s. |
| Water | ad 100.00 |

### EXAMPLE 9

Sunscreen emulsion

| | Wt. % |
|---|---|
| Caprylic acid/capric acid triglyceride | 6.00 |
| Octylmethoxy cinnamate | 5.00 |
| Butylmethoxy dibenzoylmethane | 4.00 |
| PEG 22-dodecyl copolymer | 3.00 |
| Paraffin oil (DAB9) | 2.00 |
| Cyclomethicone | 2.00 |
| Probucol | 0.50 |
| Alpha-tocopherol acetate | 0.50 |
| Na₃HEDTA | 0.50 |
| Cetyldimethicone copolyol | 0.20 |
| Preservatives, dyestuffs, perfume | q.s. |
| Water | ad 100.00 |

### EXAMPLE 10

Sunscreen emulsion

| | Wt. % |
|---|---|
| Octylmethoxy cinnamate | 4.00 |
| Castor oil | 4.00 |
| Octyl stearate | 3.00 |
| Glycerine | 3.00 |
| Cyclomethicone | 2.00 |
| Laurylmethicone copolyol | 2.00 |
| Cetylstearyl alcohol | 1.70 |
| Na₃HEDTA | 1.50 |
| Glycerol lanolate | 1.00 |
| Butylmethoxy dibenzoylmethane | 1.00 |
| Alpha-tocopherol acetate | 1.00 |
| Probucol | 0.50 |
| PEG-40 hydrogenated castor oil | 0.40 |
| Sodium cetylstearyl sulphate | 0.30 |
| Acrylamide/sodium acrylate copolymer | 0.30 |
| Hydroxypropylmethylcellulose | 0.30 |
| Caprylic acid/capric acid triglyceride | 0.10 |
| Preservatives, dyestuffs, perfume | q.s. |
| Water | ad 100.00 |

### EXAMPLE 11

Gel

| | Wt. % |
|---|---|
| Triethanolamine | 3.00 |
| Carbopol 934 P | 2.00 |
| Hydrolysed collagen | 2.00 |
| Glycerine | 2.00 |
| Sodium PCA | 0.50 |
| Probucol | 0.50 |
| Alpha-tocopherol acetate | 0.20 |
| Preservatives, dyestuffs, perfume | q.s. |
| Water | ad 100.00 |

### EXAMPLE 12

Spray formulation

| | Wt. % |
|---|---|
| Ethanol | 30.00 |
| Probucol | 0.50 |
| Alpha-tocopherol acetate | 0.10 |
| Preservatives, dyestuffs, perfume | q.s. |
| Propane/butane 25/75 | ad 100.00 |

While the invention has been described in connection with the preferred embodiment, it is not intended to limit the scope of the invention to the particular form set forth, but, on the contrary, it is intended to cover such alternatives, modifications and equivalents as may be included within the spirit and scope of the invention as defined by the appended claims.

We claim:

1. A method for treating a skin change in a patient, the method comprising:
identifying the skin change on the patient; and
applying a topical skin preparation the skin on the patient, the topical skin preparation including an active ingredient selected from the group of active ingredients including probucol, a derivative of probucol, or a combination of probucol and a derivative of probucol for treating the skin change.

2. The method as recited in claim 1 wherein the skin change includes a skin change selected from the group skin changes including an aging of the skin, a wrinkling of the skin, a change caused by an oxidative process involving the skin, a damage of the skin by ultraviolet rays and a change caused by a degenerative process involving the skin.

3. The method as recited in claim 1 wherein the topical skin preparation includes an active agent selected from a group of active agents including probucol (2,2-Propdiyl-bis(4-thio-2,6-di-tert-butylphenole), a derivative of probucol and a combination of probucol and a derivative of probucol.

4. The method as recited in claim 5 wherein the derivative of probucol is selected from the group consisting of a salt of probucol, an ester of probucol, and a protein-bound form of probucol.

5. The method as recited in claim 5 wherein the topical skin preparation further comprises at least one of a glycosaminoglycan, a derivative of a glycosaminoglycan and a hyaluronidase inhibitor.

6. The method as recited in claim 5 wherein the topical skin preparation further comprises an additive selected from the group of additives including Vitamin A, a derivative of Vitamin A, Vitamin B, a derivative of Vitamin B, vitamin C, a derivative of Vitamin C, Vitamin E, a derivative of Vitamin C, ubiquinone (CoQ10), a derivative of ubiquinone (CoQ10), idebenone, a derivative of idebenone, hyaluronic acid and a derivative of hyaluronic acid.

7. The method as recited in claim 8 wherein the hyaluronic acid and a derivative of hyaluronic acid have a molecular weight of up to 1,000,000.

8. The method as recited in claim 1 wherein the active agent has a concentration in the range of 0.0001% to 30% by weight of the preparation.

9. The method as recited in claim 1 wherein the active agent has a concentration in the range of 0.005% to 5% by weight of the preparation.

10. The method as recited in claim 1 wherein the active agent has a concentration in the range of 0.05% to 2.0% by weight of the preparation.

11. The method as recited in claim 1 wherein the topical skin preparation further comprises an ultraviolet filter substance.

12. The method as recited in claim 1 wherein the topical skin preparation further comprises at least one antioxidant agent selected from the group consisting of Vitamin E, Vitamin C, Glutathione, idebenone, Superoxide Dismutase, Catalase, Co-Enzyme Q10, Green Tea Extracts, alpha lipoic acid, and N-furfuryladenine.

13. The method as recited in claim 1 wherein the topical skin preparation further comprises an additive for reducing the formation of sunburn cells in human skin selected from the group consisting of an alpha hydroxy acid, a beta hydroxy acid, and a retinoid.

14. The method as recited in claim 1 wherein the topical skin preparation further comprises at least one depigmenting substance selected from the group consisting of kojic acid, methyl gentisate, hydroquinone, phytic acid, mulberry extract, grape seed extract, licorice extract, Vitamin A, a Vitamin A derivate, resorcinol or a derivative thereof, ellagic acid, ferulic acid, azelaic acid, an alpha hydroxyl acid and Vitamin C or derivative thereof.

15. The method as recited in claim 1 wherein the derivative of probucol further comprises a lipophilic ester of probucol.

16. The method as recited in claim 1 wherein the derivative of probucol further comprises [mono[4-[[1-[[3,5-bis(1,1-dimethylethyl)-4-hydroxyphenyl]thio]-1-methylethyl]thio]-2,6-bis(1,1-dimethylethyl)phenyl]ester] (butanedioc acid) also named AGI-1067.

17. The method as recited in claim 1 wherein the derivative of probucol further comprises [2-[4-[[1-[[3,5-bis(1,1-dimethylethyl)-4-hydroxyphenyl]thio]-1-methylethyl]thio]-2,6-bis(1,1-dimethylethyl)phenoxy]acetic acid] also named AGIX-4207.

18. The topical skin preparation as recited in claim 1 wherein the derivative of probucol is at least one of an ester of probucol and an ester of the derivative of probucol.
